(19) United States
(12) Patent Application Publication
(10) Pub. No.: US 2002/0006449 A1
(43) Pub. Date: Jan. 17, 2002

(54) Method of treatment of warts and moles

(76) Inventor: James M. Terry, Melbourne, FL (US)

Correspondence Address: MELVIN K. SILVERMAN, 4901 N. FEDERAL HWY., SUITE 410, FT. LAUDERDALE, FL, 33308, US

(21) Appl. No.: 09/810,273

(22) Filed: Mar. 19, 2001

Related U.S. Application Data
(63) Continuation-in-part of application No. 09/399,322, filed on Sep. 20, 1999, now Pat. No. 6,235,788.
(60) Provisional application No. 60/101,114, filed on Sep. 21, 1998.

(51) Int. Cl. A61K 35/78

(52) U.S. Cl. 424/760

(57) ABSTRACT

A method of use of the Capsaicin in treatment and removal of warts and moles is disclosed. The method include applying a sufficient amount of Capsaicin topically on warts and moles daily for a period time, whereby treatment, including disappearance of the warts and the moles, atrophication and de-coloration of the treated area, can be achieved. The concentration of Capsaicin is in a range from about 0.010% to about 0.200% (w/w). The Capsaicin can be applied topically through the use of a transdermal patch, or as a cream, lotion or ointment. Additionally, the Capsaicin can be used in a base of aloe vera.

### REFERENCE TO RELATED APPLICATION

This application corresponds in subject matter to that of Provisional Application Serial No. 60/101,114, filed Sep. 21, 1998, and is a Continuation-In-Part Application of co-pending patent application Ser. No. 09/399,322.

### BACKGROUND OF THE INVENTION

The botanical substance Capsaicin, a derivative of Cayenne pepper and of other types of peppers, chilies and the like, grows in different areas of the world. As such, Capsaicin is a naturally occurring substance derived from plants of the Solanaceae family, and having a chemical name of trans 8, methyl-N-vanillyl 6-nonemamide. The same, in a chemical notation, corresponds to CH₁₈H₂₇NO₃. This chemical family is known as vanilloids. Capsaicin is a white crystalline powder having a molecular weight of 305.4. It is substantially insoluble in water, however is highly soluble in alcohol, ether, and chloroform.

While to the general public, Capsaicin is simply the chemical name for various forms of cayenne peppers, the biomedical community has recognized that Capsaicin provides a mechanism of action which is of value in the treatment of rheumatoid arthritis, osteoarthritis, certain forms of neuralgias and diabetic neurophepy. Commercial forms of Capsaicin exist as arthritis creams which are applied topically to joints where painful arthritis exists. Also, U.S. Pat. No. 5,461,075 teaches the use of vaniloids in the prevention of lesions due to herpes simplex.

To the knowledge of the within inventors, no other topical use of the botanical substance Capsaicin or of vanilloids is known in the art, other than that reflected in their co-pending application Ser. No. 09/399,322. The present invention relates to a dermatological use of Capsaicin having particular application in the treatment of warts and moles of the skin, as is more fully set forth below.

### SUMMARY OF THE INVENTION

The present invention relates to a method for treating warts and moles. The method include applying a sufficient amount of Capsaicin topically on warts and moles of epithelium, whereby treatment, including disappearance of the warts and the moles, atrophication and de-coloration of treated area, is achieved. The Capsaicin can be applied through the use of a transdermal patch, and as a topical cream, lotion or ointment. Optionally, the Capsaicin can be used in a base of aloe vera.

It is an object of the present invention to provide a new use of the botanical substance Capsaicin having particular value in the treatment of dermatological conditions including warts and moles.

It is another object of the invention to provide a biomedical use of Capsaicin by which topical tumors, moles and warts can be substantially dissipated before their transformation into basal or squamous cell malignancies.

It is another object to provide a biomedical use of Capsaicin in which delivery thereof can be accomplished through the use of a transdermal patch.

The above and yet other objects and advantages of the present invention will become apparent from the hereinafter set forth Detailed Description of the Invention as set forth herein.

### BRIEF DESCRIPTION OF THE DRAWINGS

There are no drawings in this application.

### DETAILED DESCRIPTION OF THE INVENTION

Moles and warts are most common among older persons, particularly in sunbelt areas of the country, such as Florida, Texas, Arizona, and California.

The within inventors have discovered, through observations of the action of their proprietary forms of Capsaicin in the treatment of arthritis, that de-coloration and, in most cases, complete disappearance of moles and warts of the skin can be accomplished through the topical application of Capsaicin in potencies in a range of 0.010% to 0.200% (w/w).

In addition to the utility of Capsaicin with reference to age spots, the present inventors have now found that such proprietary form of Capsaicin is also effective in the removal of warts and moles.

In a further embodiment of the invention the Capsaicin may be integrated into a base of aloe vera.

As a further yet embodiment of the invention, the Capsaicin, either with or without an aloe base, may be integrated into a transdermal patch for continuous application, over a period of time to the skin. In situations where the dermatological defect is particularly deep, as in the case with a wart or mole, the use of a transdermal patch is particularly useful in that a substantial supply of the formulation can be continuously furnished over an extended period of time.

### EXAMPLE 1

Capsaicin was initially tested upon persons having lesions at joints and other areas of the body subject to arthritis. Thereby, the inventors sought out, as an initial test group, older individuals in Florida, Arizona and California, having lesions or scaling of the skin in parts of the body, typically joints, in which one or more of the above-noted Capsaicin arthritis products would otherwise be indicated for use. In this test, the inventors discovered that moles and warts in the areas being so treated for arthritis would, over a period of days of treatment, became lighter in color, atrophy, and in many cases disappear completely.

From such testing, It is believed that an appropriate range of concentration of Capsaicin is between 0.010% and 0.200% (w/w), with a preferred range being that of 0.025% to 0.099% (w/w).

### EXAMPLE 2

A Caucasian female in Stuart, Fla., experimentally utilized Capsaicin, in said preferred range, in January 1998, for the treatment of warts and, and found a complete clearing thereof within a period of weeks, following daily usage thereof.

### EXAMPLE 3

A Caucasian male in San Diego, Calif., experimentally utilized Capsaicin, in said preferred range, October 1998, for the treatment of moles on the thigh, and found a complete clearing thereof within a period of weeks, following daily usage thereof.

### EXAMPLE 4

A Caucasian female in Dallas, Tex. experimentally utilized Capsaicin, in said preferred range, in December 1998, for the treatment of warts of the foot, and found a complete clearing thereof within a period of weeks, following daily usage thereof.

### EXAMPLE 5

A Caucasian male in Bungor, Me. in January 1998 Concord, experimentally utilized the above Capsaicin formulation, in a base of aloe vera, for the treatment of pre-malignant lesions, and found a complete clearing thereof within a period of weeks, following daily usage thereof.

### EXAMPLE 6

A Caucasian female in Chicago, Ill., in February 1999 experimentally utilized the above Capsaicin formulation, in a base of aloe vera, for the treatment of pre-malignant lesions, and found a complete clearing thereof within a period of weeks, following daily usage thereof.

Thereby the inventors have found that, through such usage of Capsaicin, that warts and moles are remarkably removed from persons making use of the same.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

Having thus described our invention what I claim as new, useful, and non-obvious and, accordingly, secure by Letters Patent of the United States is:

1. A method for treating warts and moles comprising the step of:
applying a sufficient amount of Capsaicin topically on warts and moles of epithelium,
whereby treatment, including disappearance of said warts and said moles, atrophication and de-coloration of treated area, is thereby achieved.

2. The method of claim 1, wherein said Capsaicin is in a concentration from about 0.010% to about 0.200% (w/w).

3. The method of claim 1, wherein said warts include basal and squamous cell lesions.

4. The method of claim 1, wherein applying Capsaicin topically can be through the use of a transdermal patch.

5. The method of claim 1, wherein applying Capsaicin topically can be by using a roll-on stick, using topical cream, lotion or ointment, and by dabbing.

6. A method for treating warts and moles comprising the step of:
applying a sufficient amount of Capsaicin in a base of aloe vera topically on warts and moles of epithelium,
whereby treatment, including disappearance of said warts and said moles, atrophication and de-coloration of treated area, is thereby achieved.

7. The method of claim 6, wherein said Capsaicin is in a concentration from about 0.010% to about 0.200% (w/w).

8. The method of claim 6, wherein said warts include basal and squamous cell lesions.

9. The method of claim 6, wherein applying Capsaicin in a base of aloe vera topically can be through the use of a transdermal patch.

10. The method of claim 6, wherein applying Capsaicin in a base of aloe vera topically can be by using a roll-on stick, using topical cream, lotion or ointment, and by dabbing.
